(19) United States
(12) Patent Application Publication
(10) Pub. No.: US 2011/0247625 A1
(43) Pub. Date: Oct. 13, 2011

(54) BREATHING ASSISTANCE DEVICE

(76) Inventor: GEORGES BOUSSIGNAC, Antony (FR)

(21) Appl. No.: 13/084,201

(22) Filed: Apr. 11, 2011

(30) Foreign Application Priority Data
Apr. 13, 2010 (FR) 10/01555

(51) Int. Cl. A61M 16/06; A61M 16/00

(52) U.S. Cl. 128/205.25; 128/207.18

(57) ABSTRACT

The present method, system, and device relate to a breathing assistance device comprising a hollow body defining an inner chamber, provided with a breathing gas input and output, and an abutment member, intended to abut against one part of a patient and wrapped by a flexible membrane. The membrane is inserted on the outer surface of the hollow body and is able to be interposed between the abutment member and the part of the patient on which it abuts. The hollow body comprises communication ports facing to the membrane, in order to enable the breathing gas passage from the chamber to an intermediate space formed between the membrane and the outer surface of the body.

### CROSS-REFERENCE TO RELATED APPLICATION

This application claims priority to French application No. 10/01555, filed Apr. 13, 2010, entitled BREATHING ASSISTANCE DEVICE, the contents of which are expressly incorporated herein by reference.

### FIELD OF ART

The present apparatus and system are directed at a breathing assistance device usable on patients whose spontaneous breathing is absent or insufficient and method of making the same.

### BACKGROUND

From document EP 1,121,953, breathing assistance devices that enables to bring into a patient's lungs breathing gas coming from an outer source, are already known, these breathing devices comprising:
a hollow body, the inner volume of which defines a chamber provided with a breathing gas input intended to be connected to said source and with a breathing gas output intended to be connected to a respiratory tract of said patient;
an opening that constitutes said breathing gas output;
an abutment member bordering the neighbourhood of said opening contour and which is intended to abut against one part of said patient to provide seal between said breathing gas output and outside; and
a flexible membrane that completely surrounds said abutment organ.

Such known breathing assistance devices can, for instance, be in the form of:
a breathing mask, wherein said body is a hollow shell intended to be applied, through said opening which constitutes said gas output, on said patient face, enclosing the nose thereof. The breathing gas input is then formed with a tip being integral with the bottom of said shell, whereas said abutment member is in the form of a bead interposed between said opening and said patient face, or also
a nasal intubation device, wherein said hollow body is a tubular element intended to be introduced into a nostril of the patient. In such case, said breathing gas input and output are formed by the opposed ends of said tubular element and said abutment member is an inflatable bladder in the form of a balloon carried by the outer surface of said element and interposed between this one and the inner wall of said nostril.

### SUMMARY

The present method, system, and device aim at improving the above mentioned breathing assistance devices and, more particularly, completing the seal between the abutment member and the part of the patient in contact (such as, e.g., the face or the inner wall of the nostril, for instance) on which it is applied.

To this end, according to the method, system, and device, the breathing assistance device allowing to bring, in a patient's lungs, breathing gas coming from an outer source, said device comprising:
an hollow body, the inner volume of which defines an inner chamber provided with a breathing gas input intended to be connected to said source and with a breathing gas output intended to be connected to a respiratory tract of said patient;
an opening that constitutes said breathing gas output;
an abutment member bordering the neighbourhood of said opening contour and which is intended to abut against one part of said patient to provide seal between said breathing gas output and outside; and
flexible membrane that surrounds, at least partially, said abutment member, is remarkable in that:
said flexible membrane is inserted, on one of its ends, on the surface of said hollow body oriented to outside;
said flexible membrane is able to he interposed between said abutment member and the part of said patient on which it abuts; and
the hollow body comprises at least one communication port facing to said membrane, so that some breathing gas is able to cross said communication port, to circulate from said chamber in an intermediate space formed between said membrane and the outer surface of said hollow body, and to bypass the abutment member to revert then back to said inner chamber close to said opening.

Thus, thanks to the present method, system, and device, some breathing gas can cross the communication port(s) of the hollow body to circulate in the intermediate space, bypass the abutment member and finally, return back to the inner chamber close to the opening. Such breathing gas circulation leads to the formation of a gas bag between at least one portion of the abutment member and the facing flexible membrane, interposed between said portion and the part of the patient (face, nose) and tensioned by the gas circulation. This gas bag, intrinsically soft, easily fits to the patient morphology, by simple contact thereof with the concerned patient part, which provides a uniform seal between the patient part and the breathing assistance device, following the opening of the inner chamber.

Furthermore, thanks to the present method, system, and device, the application pressure of the breathing device on the abutment part of the patient can be substantially reduced to obtain the desired seal, particularly at the places where such seal cannot be obtained by an abutment member with a mere contact. The risk of sore occurrence is thus suppressed upon a prolonged wearing of the mask, while attenuating the discomfort of use for the patient.

Moreover, since the breathing gas, after having crossed the hollow body from the inner chamber, ends once more within it, no loss of breathing gas is noticed, which enables to reduce the breathing gas consumption upon the use of the breathing device.

Preferably, said hollow body comprises a plurality of communication ports evenly distributed on this latter, in order to provide a uniform distribution of the breathing gas within the intermediate space and obtain in such a way an homogeneous gas bag along the opening of the inner chamber.

Advantageously, the communication port(s) can be present appear in the form of a hole obliquely drilled in the wall of said hollow body, so that the port axis is bent with respect to the local direction orthogonal to said wall. Thus, the breathing gas passage is facilitated in the intermediate space, while reducing disturbances in the neighborhood on the communication port(s) in the inner chamber.

Preferably, said flexible membrane is made in a plastic film (for instance silicone) of some micrometers of thickness.

In an embodiment according to the present method, system, and device, the breathing device is in the form of a nasal or bucconasal mask, wherein said body is a hollow shell intended to be applied, through said opening, on a patient's face enclosing at least his/her nose, the breathing gas input being provided in the bottom of said shell and said abutment member being in the form of a bead following the contour of said shell opening and interposed between said opening and said patient's face.

Thus, when the mask is applied on the patient's face, the gas bag is distorted to fit and comply, by adjustment, with the morphology of the patient's face and, particularly, with its irregularities, which enables the correction of the bead sealing defects without resorting to an important application pressure of the mask on the face. Furthermore, one can easily adapt a breathing mask of given dimensions (preferably intended for large faces) on any kind of face, whatever the dimensions thereof are.

Preferably, the other end of said membrane is free and extends to the inside of said opening. Thus, the breathing gas circulation between the membrane and the bead enables to press the free end against the face, which increases the membrane portion in contact with the face and, as a result, improves the sealing.

In a variation of the above mentioned embodiment according to the present method, system, and device, the other end of said membrane can be inserted on the surface of said hollow shell facing said chamber, between said bead and said communication port, and at least a discharge port can be arranged in the portion of said membrane located inside said chamber, in order to enable the breathing gas discharge in this latter.

Furthermore, in order to insure a permanent swelling of a flexible membrane, preferably the section of said communication port is larger than said discharge port one.

In another embodiment of the present method, system, and device, the breathing device is in the form of a nasal intubation device, wherein said hollow body is a tubular element intended to be introduced inside a nostril of the patient, said breathing gas input and output being formed by the opposed ends of said tubular element and said abutment member being in the form of a balloon brought by the outer surface of said tubular element and interposed between this one and the inner wall of said nostril.

Preferably, said membrane completely covers said abutment member and extends, by its other free end, to the interior of said opening.

Furthermore, whatever the embodiment of the breathing assistance device according to the present method, system, and device, the flexible membrane can be removably inserted on said hollow body.

Additional examples and details of the present method, system, and device are also described in the following detailed description, drawings, and appended claims.

Various embodiments of the present method, system, and device are described in detail in the detailed description and claims below. Any feature or combination of features described herein are included within the scope of the present method, system, and device and in combination whether expressly described provided that the features included in any such combination are not mutually inconsistent as will be apparent from the context of the described features and knowledge of one of ordinary skill in the art. In addition, any feature or combination of features may be specifically excluded from any embodiment of the present method, system, and device.

### BRIEF DESCRIPTIONS OF THE FIGURES

The Figures of the attached drawing will help to understand how the present method, system, and device can be performed. On these figures, like reference numerals relate to like components.

FIG. 1 schematically shows, according to an axial section, an examplary breathing assistance device according to the present method, system, and device, being in the form of a mask.

FIGS. 2 and 3 schematically illustrate, in enlarged partial views, the process according to which the sealing is obtained with the mask of FIG. 1.

FIG. 4, like FIG. 1, illustrates another examplary breathing mask according to the present method, system, and device.

FIG. 5 schematically shows, in axial section, an alternative embodiment of the breathing assistance device according to the present method, system, and device, being in the form of a tubular element.

### DETAILED DESCRIPTION

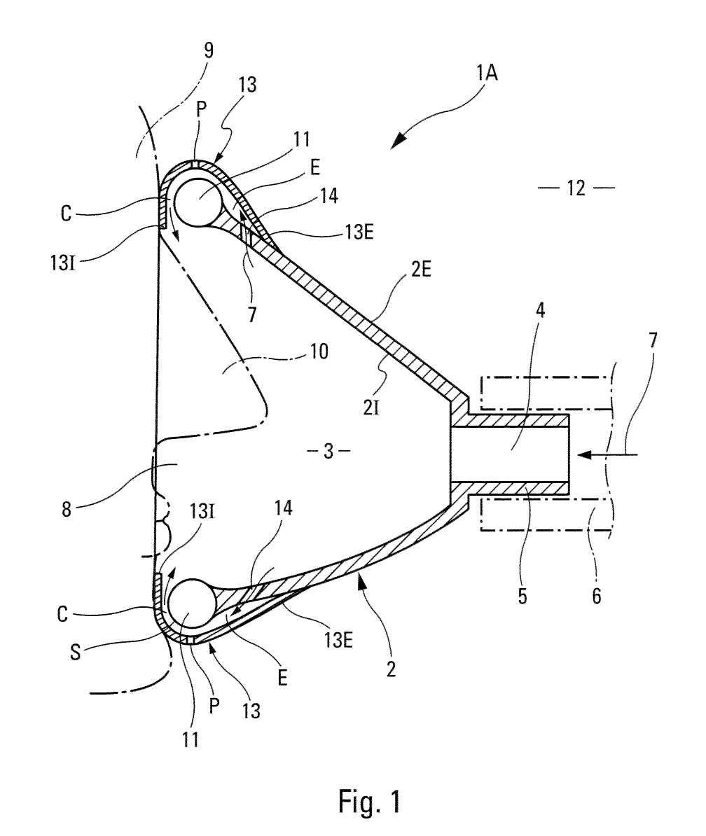

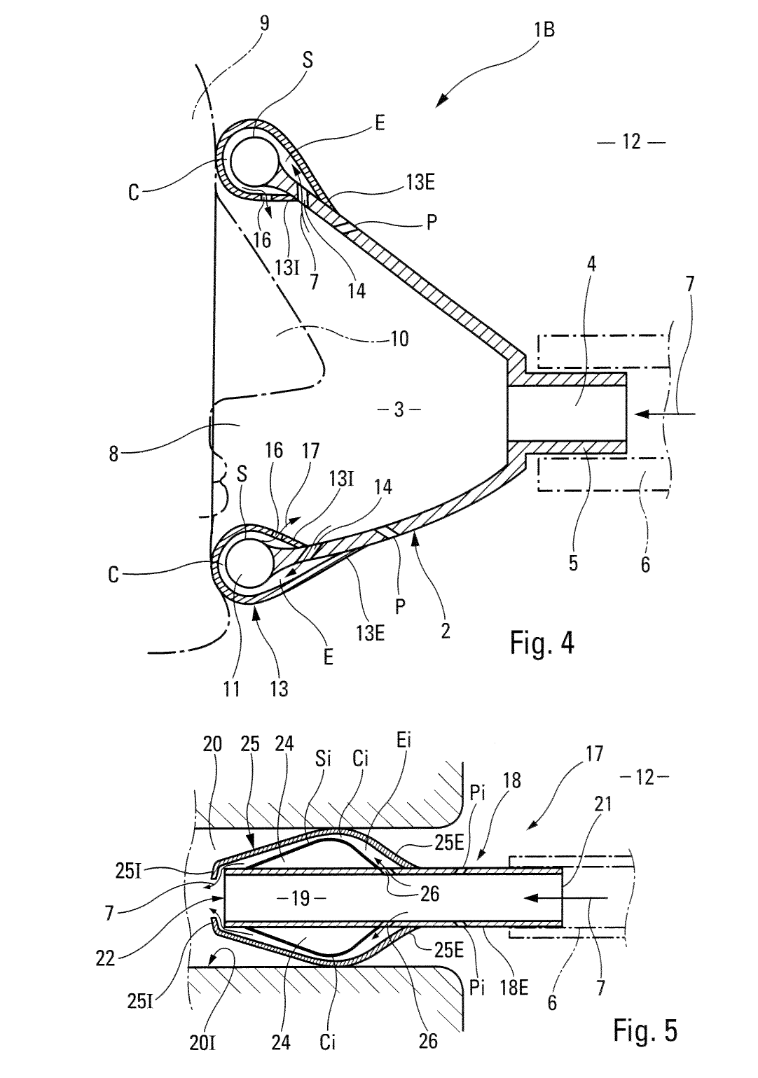

The breathing assistance device 1A, 1B, according to the present method, system, and device and represented on FIGS. 1 and 4, is shown in the form of a breathing mask comprising an rigid hollow shell 2 defining an inner chamber 3. At the bottom of the shell 2 a breathing gas input 4 is arranged, for instance thanks to a tubular tip 5, being integral with said shell 2, that may be connected to a breathing gas source (not illustrated), for instance a pressurized bottle, by an appropriate tubing 6. On FIGS. 1 and 4, the arrival of fresh breathing gas is symbolized by the arrow 7.

The inner chamber 3 comprises a breathing gas outlet constituted by the opening 8 of said rigid shell 2. This one is intended to be applied, through its opening 8, on a patient's face 9 (shown in mixed lines) enclosing his/her nose 10.

In order to provide sealing to the gas between the opening 8 of the shell 2 and the face 9, the breathing mask 1A, 1B comprises an abutment member in the form of a bead 11, being integral with the shell 2 and following the contour of the opening 8 of this latter. The bead 11 is interposed between said opening 8 and the face 9 of the patient, when the shell 2 is applied against the face 9 for isolating the inner chamber 3 from the outside 12.

Moreover, the bead 11 bordering the opening 8 is made of a flexible foam, preferably with closed cells. Alternatively, it could be present in the form of a thin wall inflatable bladder.

The breathing mask 1A, 1B also comprises a flexible membrane 13 in the form of a plastic film of a few microns of thickness, which surrounds at least partially said bead 11. Thus, the membrane 13 is more flexible than the foam bead 11.

According to the present method, system, and device, the flexible membrane 13 is inserted (for instance by gluing) at one of its ends 13E, on the external surface 2E of the hollow shell 2. When the mask 1A, 1B is located on the face 9 of the patient, the flexible membrane 13 is interposed between the bead 11 and the face 9 on which it is abutted.

Furthermore, the hollow shell 2 comprises a plurality of communication ports 14 facing o the membrane 13, in order to enable the breathing gas 7 passage from the inner chamber 3 to an intermediate space E formed between the membrane 13 and the outer surface 2E of the hollow shell 2. The intermediate space E further extends between the membrane 13 and at least one part of the bead 11 surface S.

Preferably, the communication ports 14 are regularly distributed around the hollow shell 2, in order to ensure an even distribution of breathing gas 7 in the intermediate space E.

Each one of the ports 14 is advantageously in the form of an obliquely drilled hole in the wall of the shell 2, so that the axis of said port 14 is bent based on the orthogonal local direction with respect to the wall portion of the shell 2, in order to facilitate the breathing gas 7 coming in the space E.

In the embodiment illustrated on FIGS. 1 to 3 the other end 131 of the membrane 13 is free and extends to the inside of the opening 8.

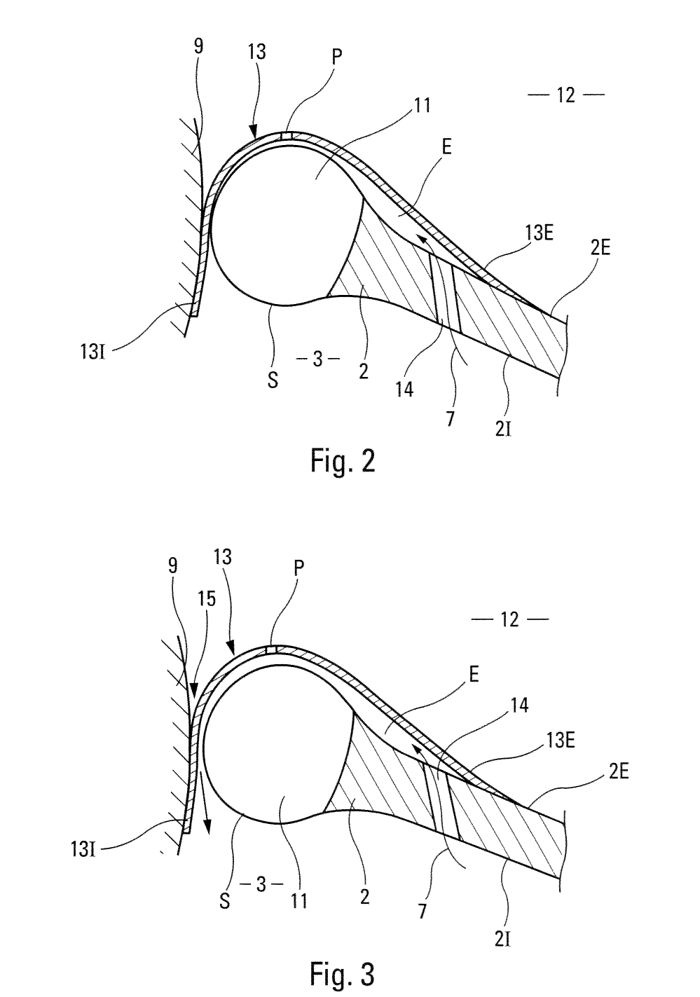

When the mask 1A is applied on the face 9 of the patient, the bead 11, due to its relative rigidity and to irregularities of reliefs of the face 9, cannot be evenly applied on this latter in a strictly sealed way. Certainly, on the great part of the mask 1A contour, the bead 11 sealingly applies against the face 9, as illustrated on FIG. 2, so that the membrane 13 being interposed is itself pressed between the bead 11 and the face 9. On the contrary, in certain places of said bead 11, this one is maintained distant from the face 9, letting still some space 15 with this latter (see FIG. 3).

In this last case, a part of the breathing gas 7, penetrating into the rigid shell 2 through the opening 4 (arrow 7) and entering the inner chamber 3, Ewes through the communication ports 14 to circulate in the intermediate space E, bypassing the bead 11 and ending again in the inner chamber 3. The gas circulation 7 in the intermediate space E leads to the formation of a gas bag C between at least one portion of the bead 11 surface S and the facing membrane 13 tensioned by the gas circulation in the space E. This soft gas bag C easily fits to the patient morphology, by simple contact thereof with the patient's concerned part, which ensures a uniform seal between the face 9 and the mask 1A following the opening 8. Thus, the membrane 13 applies against the face 9 of the patient by obstructing the space 15.

Consequently, the flexible membrane 13 enables to complete the sealing of the breathing assistance mask 1A, on the places where this sealing cannot be ensured by the bead 11.

Moreover, since the membrane 13 is flexible and elastic and it presents a free end 131 in the opening 8, the breathing mask can automatically fit the different morphologies of faces of different dimensions, the tensioned flexible membrane 13 fittingly conforming on those faces.

Furthermore, during the gas circulation 7 in the intermediate space E, the membrane 13 free end 131 is pressed against the face 9, which optimizes the sealing increasing the contact surface between the membrane 13 and the face 9. The open annular area formed between the membrane 13 free end 131 and the facing bead 11 defines a gas discharge means.

It should be noticed that, as shown on FIGS. 1 to 3, the mask 1A can comprise one or more perforations P, drilled on the membrane 13 and intended to enable the discharge of polluted gas expired by the patient. Alternatively or complementarily, these perforations could be arranged in the hollow shell 2, between the membrane 13 end 13E and the gas input 4 (see FIG. 4).

In the other examplary mask 1B of the present method, system, and device illustrated on FIG. 4, the other end 131 of the membrane 13 is inserted on the inner surface 21 of the hollow shell 2 facing the chamber 3. It is fastened, for instance by gluing, between the bead 11 and the communication ports 14, so that the latter are not covered by the membrane portion 13 placed inside the chamber 3.

As shown on FIG. 4, several discharge ports 16 are arranged in the membrane portion 13 inside the chamber 3, in order to enable an evacuation, in this latter, of the breathing gas 7 having previously circulated in the intermediate space E.

In case the communication ports 14 and discharge ports 16 are in the same number, the section of the communication ports 14 is advantageously larger than that of the discharge ports 16, in order to ensure a permanent swelling of the flexible membrane 13.

The process according to which the sealing is obtained with the mask 1B of FIG. 4 is similar to that described facing the mask 1A of FIGS. 1 to 3.

In an alternative embodiment schematically illustrated by FIG. 5, the breathing assistance device 17 according to the present method, system, and device is in the form of a nasal intubation device comprising a tubular element 18, the inner volume of which defines an inner chamber 19, which is intended to be introduces into a nostril 20 of a patient. Breathing gas 7, coming from an outer source not represented, is introduced by a tubing 6 in the tubular element 18 through a proximal end 21 of this latter and is assigned to the lungs of said patient through the distal end 22 of said element 18, arriving in the nostril 20.

The device 17 comprises an abutment member 24 being in the form of a supporting and sealing balloon, able to abut against the inner wall 20I of the nostril 20. The balloon 24 is supported by the outer surface 18E of the tubular element 18 and is interposed between this latter and the inner wall 20I.

As illustrated on FIG. 5, the balloon 24 surface Si is covered by a flexible membrane 25. This membrane 25 comprises an end 25E being inserted (for instance by gluing) on the outer surface 18E of the tubular element 18 and a free inner end 25I which extends toward said opening formed by the distal end 22 of the tubular element 18.

Furthermore, the tubular element 18 comprises a plurality of communication ports 26 facing to the membrane 25, so that breathing gas 7 can he transferred from the inner chamber 19 to an intermediate space Ei. This space Ei is formed between the membrane 25 and the outer surface 18E of the element 18 and extends between the membrane 25 and the surface Si of the balloon 24.

The ports 26, advantageously equi-angularly distributed on the same section of the tubular element 18, are obliquely bent with respect to the orthogonal local direction of the tubular element wall 18.

Thus, the breathing gas 7, coming from the source, can go through the ports 26 and circulate in the intermediate space Ei, by bypassing the balloon 24, which swells the membrane 25 and forms a gas bag Ci able to fit the various irregularities of the inner wall 20I of the patient nostril 20.

It will be easily understood that, similarly to what has been explained above facing to FIGS. 1 to 4, the membrane 25 is able to ensure the sealing between the inner wall 20I of the nostril 20 and the output 22 of the tubular element 18, on the places w here the balloon 24 presents sealing defects. The open annular area formed between the membrane 25 free end 25I and the tubular element 18 defines a gas discharge means.

Furthermore, one or more perforations Pi can be arranged in the wall of the tubular wall 18, between the end 25E of the membrane 25 and the proximal end 21, in order to perform a polluted gas discharge rejected by the patient and circulating inside the chamber 19.

Although the disclosure herein refers to certain specific embodiments, it is to be understood that these embodiments are presented by way of example and not by way of limitation. The intent of the foregoing detailed description, although discussing exemplary embodiments, is to be construed to cover all modifications, alternatives, and equivalents of the embodiments as may fall within the spirit and scope of the present method, system, and device as defined by the claims.

What is claimed is:

1. A breathing assistance device to supply breathing gas coming from an outer source, said device comprising:
a hollow body, the inner volume of which defines an inner chamber provided with a breathing gas input intended to be connected to said source and with a breathing gas output intended to be connected to a respiratory tract of said patient:
an opening that constitutes said breathing gas output;
an abutment member bordering the neighborhood of said opening contour and which is intended to abut against one part of said patient to provide seal between said breathing gas output and outside; and
a flexible membrane which wraps at least partially said abutment member, wherein:
said flexible membrane is inserted, on one of its ends, on the surface of said hollow body oriented to the outside;
said flexible membrane is able to be interposed between said abutment member and the part of said patient on which it abuts; and
the hollow body comprises at least one communication port facing to said membrane,
wherein some breathing gas is able to cross said communication port, to circulate from said chamber in an intermediate space formed between said membrane and the outer surface of said hollow body, and to bypass the abutment member to revert back to said inner chamber close to said opening.

2. The device according to claim 1, wherein said hollow body comprises a plurality of communication ports regularly distributed on the hollow body.

3. The device according to claim 1, wherein said communication port is in the form of a hole obliquely drilled in the wall of said hollow body, so that the axis of said port is bent with respect to the local direction orthogonal to said wall.

4. The device according to claim 1, wherein said flexible membrane is made of a plastic film of some micrometers of thickness.

5. The device according to claim 2, wherein it is in the form of a mask, wherein said body is a hollow shell intended to be applied, through said opening, on a patient's face, enclosing at least his/her nose, the breathing gas input being provided in the bottom of said shell and said abutment member being in the form of a bead following the contour of said shell opening and interposed between said opening and said patient face.

6. The device according to claim 5, wherein the other end of said membrane is free and extends inside said opening.

7. The device according to claim 5, wherein:
the other end of said membrane is inserted on the surface of said hollow shell facing said chamber, between said bead and said communication port; and
at least one discharge port is arranged in the portion of said membrane arranged inside said chamber, in order to enable a discharge of breathing gas in the chamber.

8. The device according to claim 7, wherein the section of said communication port is larger than that of said discharge port.

9. The device according to claim 1, wherein it is in the form of a nasal intubation device, wherein said hollow body is a tubular element intended to be introduced in a nostril of the patient, said breathing input and output being formed by the opposed ends of said tubular element and said abutment member being in the form of a balloon supported by the outer surface of said tubular element and interposed between this one and the inner wall of said nostril.

10. The device according to claim 9, wherein said membrane completely corers said abutment member and extends, by its other free end, to the inside of said opening.
